(19) United States
(12) Patent Application Publication
(10) Pub. No.: US 2024/0188688 A1
(43) Pub. Date: Jun. 13, 2024

(54) MEHTOD AND APPARATUS FOR PROCESSING FOOT INFORMATION

(71) Applicant: PERFITT INC., Daejeon (KR)

(72) Inventors: Steena Sun Yong LEE, Seoul (KR); Hyun Woo NAM, Seoul (KR); Jae Hun CHO, Seoul (KR)

(21) Appl. No.: 18/587,801

(22) Filed: Feb. 26, 2024

Related U.S. Application Data
(63) Continuation-in-part of application No. 18/241,985, filed on Sep. 4, 2023.
Application No. 18/241,985 is a continuation of application No. 16/979,111, filed on Sep. 8, 2020, now Pat. No. 11,779,084.
(63) Continuation of application No. PCT/KR2022/014895, filed on Oct. 4, 2022.

(30) Foreign Application Priority Data
Mar. 9, 2018 (KR) 10-2018-0028311
Nov. 4, 2021 (KR) 10-2021-0150726

(51) Int. Cl. A43D 1/02; A61B 5/107; G06T 7/00; G06T 7/60; G06T 7/73

(52) U.S. Cl. CPC A43D 1/025; A61B 5/1074; G06T 7/0012; G06T 7/60; G06T 7/73

(57) ABSTRACT

A method for measuring foot size and shape by using image processing, according to one embodiment of the present invention, comprises: a step of acquiring an image captured by simultaneously photographing a user's foot and an item having a standardized size; and a calculation step of calculating foot size or shape information from the image. The image is captured when at least a part of the user's foot comes in contact with the item.

The present disclosure relates to a method and apparatus for processing foot information and recommending a type and size of shoes, based on three-dimensional (3D) scanning of a foot.

### CROSS REFERENCE TO RELATED APPLICATIONS

This application is a Continuation-in-Part Application of U.S. patent application Ser. No. 18/241,985, filed on Sep. 4, 2023, which is a Continuation Application of U.S. Pat. No. 11,779,084 filed on Sep. 8, 2020, by application Ser. No. 16/979,111, which is a National Stage Entry of International Patent Application No. PCT/KR2019/002799, filed on Mar. 11, 2019, which claims priority from and the benefit of Korean Patent Application No. 10-2018-0028311, filed on Mar. 9, 2018, each of which is hereby incorporated by reference for all purposes as if fully set forth herein. This application is also a Bypass Continuation of International Patent Application No. PCT/KR2022/014895, filed on Oct. 4, 2022, which claims priority from and the benefit of Korean Patent Application No. 10-2021-0150726, filed on Nov. 4, 2021, each of which is hereby incorporated by reference for all purposes as if fully set forth herein.

### BACKGROUND

### Field

The present disclosure relates to a method for measuring foot size and/or shape by using image processing.

The present disclosure relates to a method and apparatus for processing foot information and recommending a type and size of shoes, based on three-dimensional (3D) scanning of a foot.

### Discussion of the Background

In most shoe manufacturing industries, shoes are manufactured by classifying only the size of the foot at 5 mm intervals without classifying the shape of the foot. There is no standardized dimension standard for each company, and even with shoes of the same size, shoes of different brands sometimes do not fit the feet. In the case of wearing shoes that do not fit the feet like this, symptoms of foot deformation such as hallux valgus may occur due to continuous compression of the shoes, and such deformation of the feet may damage the overall health of the body.

Recently, in order to prevent this risk, the technology in which the shape of a user's foot is three-dimensionally (3D) scanned to manufacture shoes considering several requirements such as the size of the user's foot, the arch, the width of feet, the toe length and the height of the instep of the foot so as to produce shoes that fit exactly to the user's foot, has been developed and used.

Existing foot measurement methods for purchasing custom-made shoes include methods in which a customer manually inputs and transmits foot measurements such as left- and right-foot lengths and widths, which are items related to his or her foot size, or methods using an expensive 3D foot measuring apparatus, but the reliability of values manually measured by customers may be poor. In addition, when using a foot measuring apparatus, there is the inconvenience of having to visit a store where the foot measuring apparatus is available.

The above-mentioned background art is technical information possessed by the inventor for the derivation of the present disclosure or acquired during the derivation of the present disclosure, and cannot necessarily be said to be a known technique disclosed to the general public prior to the filing of the present disclosure.

### SUMMARY

Provided is a method for measuring foot size and/or shape by using image processing.

An objective of the present disclosure is to reduce the time and cost of visiting a store in person to measure a foot size.

An objective of the present disclosure is to facilitate the measurement of foot dimensions and improve the accuracy of foot dimensions.

An objective of the present disclosure is to recommend a type and size of suitable shoes for foot dimension information.

According to an aspect of the present disclosure, a method for measuring foot size and shape by using image processing, includes acquiring an image captured by simultaneously photographing a user's foot and an item having a standardized size and calculating foot size or shape information from the image. The image may be captured when at least a part of the user's foot comes in contact with the item.

The item may have a rectangular shape, and the image may be photographed when all of four vertices of the item are exposed and the user's foot covers a part of edges of the item.

The calculating of the foot size or shape information from the image may include calculating positions of vertices of the item from the image and calculating a region where the foot is located in the image.

The calculating of the positions of the vertices may include calculating one or more feature points corresponding to a corner from the image, calculating a convex polygon that surrounds a contour of the item from the image and then calculating candidate points of the vertices of the item through a simplification algorithm, and comparing the one or more feature points with the candidate points to select the vertices.

The calculating of the region where the foot is located in the image, may include calculating a first region including a region where the foot and the item do not come in contact with each other, inside a figure formed by the vertices and removing the first region from the image and then calculating a contour of the other region.

The calculating of the foot size or shape information from the image may include calculating a difference between relative lengths of toes from the image to determine a shape type of the foot.

The method may further include, before the acquiring of the image, providing a guide interface to photograph the image when a part of the user's foot comes in contact with the item.

The calculating of the foot size or shape information from the image may include calculating a region where the foot is located in the image and measuring an angle of hallux valgus of the foot from the calculated foot region.

According to another aspect of the present disclosure, there is provided an application program combined with hardware and stored in a computer-readable recording medium to implement the method described above.

Other aspects, features, and advantages than those described above will become apparent from the claims and a detailed description of the present disclosure.

A method of processing foot information according to an embodiment of the present disclosure is performed by a processor of an apparatus for processing foot information, and includes generating an initial three-dimensional (3D) model of a foot based on a plurality of foot images, which are obtained by photographing the foot from various angles, generating an intermediate 3D model by removing ground and noise from the initial 3D model, generating a final 3D model with a normal foot shape by restoring a portion of the foot that has been removed along with the ground when generating the intermediate 3D model, and calculating foot-related information from the final 3D model and recommending shoes.

An apparatus for processing foot information according to an embodiment of the present disclosure includes a processor, and a memory operatively connected to the processor and storing at least one piece of code to be executed by the processor, wherein the memory stores code that, when executed by the processor, causes the processor to generate an initial 3D model of a foot based on a plurality of foot images, which are obtained by photographing the foot from various angles, generate an intermediate 3D model by removing ground and noise from the initial 3D model, generate a final 3D model with a normal foot shape by restoring a portion of the foot that has been removed along with the ground when generating the intermediate 3D model, calculate foot-related information from the final 3D model, and recommend shoes.

In a method for measuring foot size and shape by using image processing according to an embodiment of the present disclosure, a user can measure foot size and/or shape automatically/semi-automatically through an image captured by a user through photographing. Thus, the user can not only easily know information about his/her own foot size conveniently, but also can receive information about shoes that do fit his/her own feet. Of course, the scope of the present disclosure is not limited by these effects.

According to the present disclosure, it is possible to provide convenience to a user by reducing the time and cost of visiting a store in person to measure foot dimensions.

In addition, by performing three-dimensional (3D) scanning using a smartphone, foot dimensions may be easily performed and the accuracy of the foot dimensions may be improved.

In addition, it is possible to help a user purchase shoes by recommending a type and size of shoes suitable for foot dimension information.

### BRIEF DESCRIPTION OF THE DRAWINGS

FIG. 1 is a time-sequential flowchart illustrating operations of a method for measuring foot size and shape by using image processing according to an embodiment of the present disclosure, and FIGS. 2 and 3 are diagrams schematically showing an image used in the method for measuring foot size and shape by using image processing according to an embodiment of the present disclosure.

FIG. 4 is a flowchart illustrating a calculating operation according to an embodiment.

FIG. 5 is a diagram showing an operation of calculating feature points.

FIGS. 6A through 6C are diagrams sequentially illustrating an operation of calculating candidate points of vertices of an item.

FIG. 7 is a diagram illustrating an operation of selecting vertices of an item.

FIGS. 8A through 8C are diagrams sequentially illustrating an operation of calculating a region where the foot is located in an image, according to an embodiment.

FIGS. 9A and 9B are diagrams illustrating a method for measuring the size of a foot according to an embodiment.

FIG. 10A through 10E are diagrams illustrating various toe shape types.

FIG. 11 is a time-sequential flowchart illustrating operations of a method for measuring foot size and shape by using image processing according to an embodiment of the present disclosure.

FIG. 12 is a diagram illustrating an interface for guiding a photographing method.

FIG. 13 is a time-sequential flowchart illustrating operations of a method for measuring foot size and shape by using image processing according to an embodiment of the present disclosure

FIG. 14 is a diagram illustrating an interface for providing information about foot and shoes.

FIG. 15 is a time-sequential flowchart illustrating operations of a method for measuring foot size and shape by using image processing according to an embodiment of the present disclosure.

FIG. 16 is a diagram illustrating a method for measuring an angle of hallux valgus.

FIG. 17 is a diagram of an example of a foot information processing environment including a foot information processing apparatus, an electronic apparatus, and a network connecting them to each other, according to an embodiment.

FIG. 18 is a diagram of an example of a foot information processing environment according to another embodiment.

FIG. 19 is a block diagram schematically illustrating a configuration of a foot information processing apparatus according to an embodiment.

FIG. 20 is a block diagram for schematically describing a configuration of a foot information processing unit according to an embodiment.

FIGS. 21 through 24 are diagrams of examples for describing foot information processing according to an embodiment.

FIG. 25 is a block diagram schematically illustrating a configuration of a foot information processing apparatus according to another embodiment.

FIG. 26 is a flowchart for describing a foot information processing method according to an embodiment.

### DETAILED DESCRIPTION

As the present disclosure allows for various changes and numerous embodiments, particular embodiments will be illustrated in the drawings and described in detail in the written description. The effects and features of the present disclosure, and ways to achieve them will become apparent with reference to embodiments to be described later in detail together with the drawings. However, the present disclosure is not limited to the following embodiments but may be implemented in various forms.

It will be understood that although the terms “first,” “second,” etc. may be used herein to describe various components, these components should not be limited by these terms.

As used herein, the singular forms “a,” “an,” and “the” are intended to include the plural forms as well, unless the context clearly indicates otherwise.

It will be further understood that the terms “comprises” and/or “comprising” used herein specify the presence of stated features or components, but do not preclude the presence or addition of one of more other features or components.

It will be further understood that when a layer, region, or component is referred to as being “formed on,” another layer, region, or component, it can be directly or indirectly formed on the other layer, region, or component. That is, for example, intervening layers, regions, or components may be present.

When a certain embodiment may be implemented differently, a specific process order may be performed differently from the described order. For example, two consecutively described processes may be performed substantially at the same time or performed in an order opposite to the described order.

Sizes of elements in the drawings may be exaggerated for convenience of explanation. In other words, since sizes and thicknesses of components in the drawings are arbitrarily illustrated for convenience of explanation, the following embodiments are not limited thereto.

In the present specification, ‘foot size information’ or ‘shape information’ should be interpreted as a concept including all kinds of information about a user's foot. For example, ‘foot size information’ or ‘shape information’ refers to quantitative information about the length, the angle, the width, etc. such as the length of the foot, the width of feet, a distance between a specific point on the foot and another point, the width of the foot outline, the angle of three specific points of the foot, and/or qualitative information such as the shape of the outline of the foot, the shape type of the foot, the presence or absence of a foot-related disease, and the degree of the disease, but the ‘information’ of the present disclosure is not limited thereto.

A method for measuring foot size and shape by using image processing according to an embodiment of the present disclosure may be implemented by a measuring apparatus (not shown). The measuring apparatus may correspond to at least one processor or may include at least one processor. Thus, the measuring apparatus may be driven in a form included in other hardware devices such as a microprocessor, a general-purpose computer system, a tablet, or smartphone.

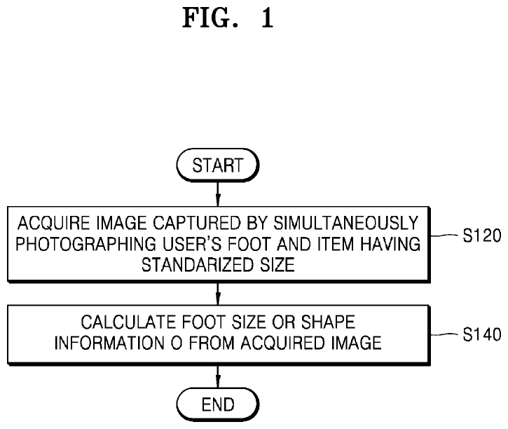

FIG. 1 is a time-sequential flowchart illustrating operations of a method for measuring foot size and shape by using image processing according to an embodiment of the present disclosure, and FIGS. 2 and 3 are diagrams schematically showing an image IMG used in the method for measuring foot size and shape by using image processing according to an embodiment of the present disclosure. FIGS. 2 and 3 illustrate a state in which there are no other objects than an item 10 and a foot F in a background B of the image IMG for convenience of explanation.

The method for measuring foot size and shape by using image processing according to an embodiment of the present disclosure may include acquiring the image IMG captured by simultaneously photographing the user's foot F and the item 10 having a standardized size (S120) and calculating size or shape information of the foot F from the image IMG (S140).

Referring to FIGS. 1, 2 and 3, acquiring of the image IMG captured by simultaneously photographing the user's foot F and the item 10 having the standardized size (S120) may be first performed. The item 10 having the standardized size may be a planar object commonly used in real life such as a paper, an envelope, a notebook, a sketch book, and a file. Hereinafter, it will be described as an example in which the item 10 having the standardized size is an A4 paper having the size of 210 mm×297 mm.

Since, in an embodiment of the present disclosure, foot size and shape is measured by using image processing, the image IMG captured by photographing the foot F is essentially used. Although FIGS. 2 and 3 illustrate the image IMG captured by photographing the bare foot F, the image IMG captured by photographing the foot F in socks may also be used. The item 10 having the standardized size may provide a reference for calculating/acquiring size information from the captured image IMG. Thus, the item 10 and the foot F may be simultaneously photographed.

In the present disclosure, the image IMG captured by photographing the user's foot F and the item 10 when at least a part of the user's foot F comes in contact with the item 10, may be image-processed so that the user's foot size may be measured. When the image IMG captured by simultaneously photographing the foot F and the item 10 in the state in which the foot F and the item 10 come in contact with each other, is used, the foot F and the item 10 may be easily distinguished from each other within the image IMG through an image processing algorithm using a color value difference.

In an embodiment, the acquired image IMG may be photographed when all of four vertices of the item 10 are exposed and the user's foot F covers a part of edges of the item 10.

Referring to FIG. 2, the image IMG captured in a state in which the user's foot F covers a part of the edges of a vertical length of the A4 paper, is shown. The image IMG used in image analysis may be captured in a state in which both ends of the foot F come in contact with the edges of the A4 paper.

In an embodiment of the present disclosure, after coordinates of four vertices C₁, C₂, C₃, and C₄ of the item 10 are checked within the image IMG, affine transformation may be performed to be suitable for the original size and shape of the item 10 so that a size reference may be acquired to calculate the size of the foot F within the image IMG. Thus, in the acquired image IMG, all of the four vertices C₁, C₂, C₃, and C₄ of the item 10 may be exposed.

In addition, the image IMG may be captured when the foot F covers a part of edges L₁₂, L₂₃, L₃₄, and L₄₁ of the item 10. If the image IMG is captured in a state in which the foot F is located in the middle of the item 10, a part of the vertices C₁, C₂, C₃, and C₄ of the item 10 may not be seen from the image IMG due to the leg L photographed together with the foot F. In this case, affine transformation, which is a prerequisite for securing the size reference, is not capable of being performed. Thus, the image IMG used in image processing may be captured in a state in which the foot F covers a part of the edges of the item 10 so that all of the four vertices C₁, C₂, C₃, and C₄ of the item 10 may be seen.

According to an embodiment, the image IMG may be captured in a state in which both ends of the foot F come in contact with vertical edges of the item 10. Referring to FIG. 2, the image IMG captured in a state in which a right vertical edge L₂₃ of four edges of the A4 paper comes in contact with the foot F, is shown. Both ends, i.e., the tip of a toe end and the tip of the heel of the foot F may come in contact with the edges. In this case, the four vertices C₁, C₂, C₃, and C₄ may be recognized within the image IMG. In the case of using the image IMG shown in FIG. 2, length information of the foot F may be easily calculated through image processing.

According to an embodiment, the image IMG may be captured in a state in which both ends of the feet come in contact with horizontal edges of the item 10. Referring to FIG. 3, the image IMG captured in a state in which a lower horizontal edge L₃₄ of four edges of the A4 paper comes in contact with the foot F, is shown. Both ends of the feet, i.e., a left end and a right end of the foot F, may come in contact with the edges. In this case, all of the four vertices C₁, C₂, C₃, and C₄ may be recognized within the image IMG. When the image IMG shown in FIG. 3 is used, information about the width of the foot F (the length of feet) may be easily calculated through image processing.

The acquired image IMG may be pre-processed. A pre-processing operation may include a black and white image conversion operation, an adaptive sharpening operation, a blurring operation, an adaptive threshold filter application operation, and a pre-processing method is not limited thereto.

Hereinafter, a method of calculating information about the size or shape of the foot from the image IMG through image processing will be described.

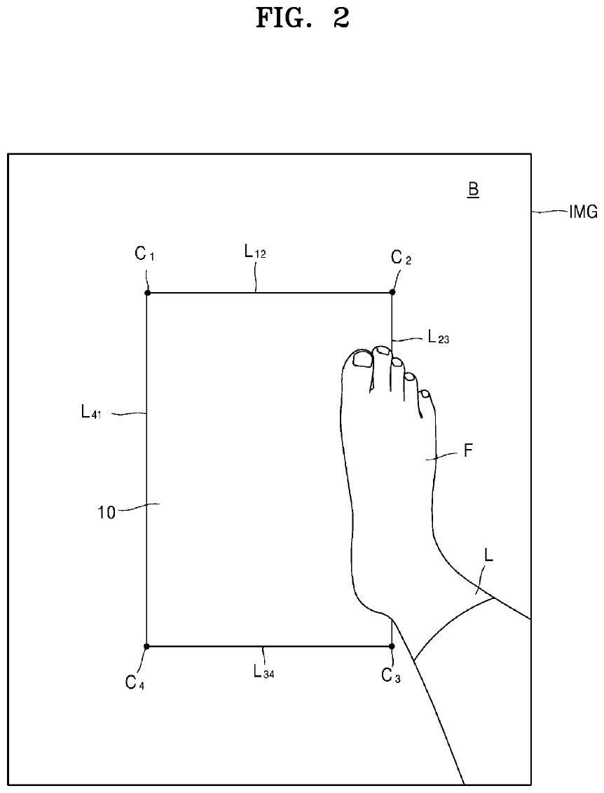

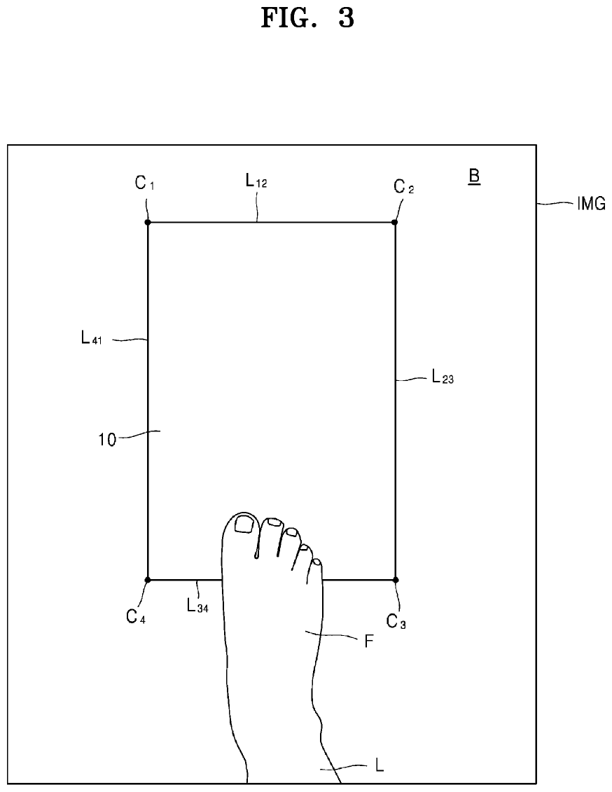

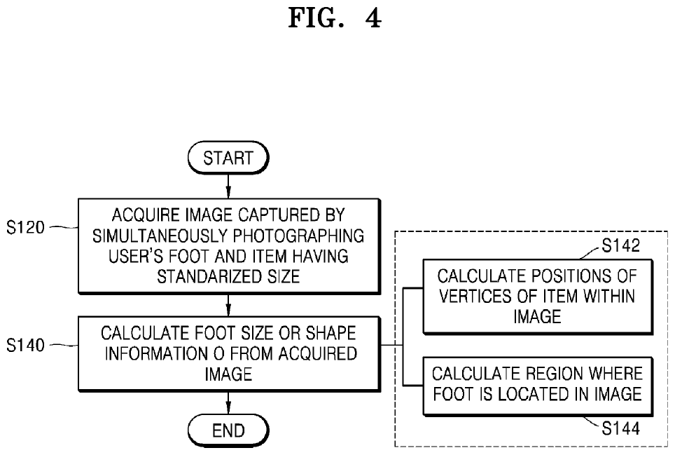

FIG. 4 is a flowchart illustrating a calculating operation (S140) according to an embodiment. The calculating operation (S140) according to an embodiment may include calculating positions of vertices of the item 10 within the image IMG (S142) and calculating a region where the foot F is located in the image IMG (S144).

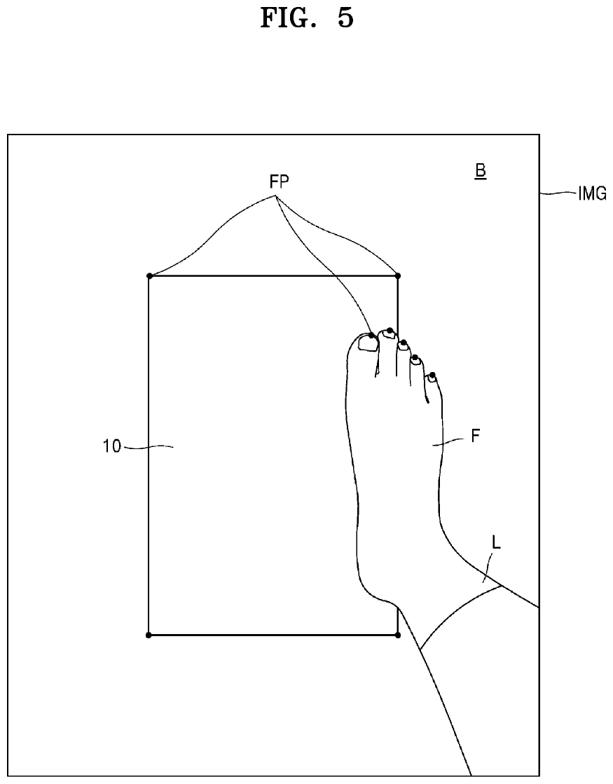

First, the calculating of the positions of the vertices of the item 10 within the image IMG will be described with reference to FIGS. 5, 6A, 6B, 6C, and 7.

The calculating of the positions of the vertices of the item 10 (S142) according to an embodiment may include calculating feature points, calculating candidate points of vertices, and selecting vertices.

FIG. 5 is a diagram illustrating an operation of calculating feature points FP. According to an embodiment, the calculating of the positions of the vertices may include calculating one or more feature points FP corresponding to a corner from the image IMG. Referring to FIG. 5, calculating of feature points FP to be determined as a ‘corner’ from the (pre-processed) image IMG may be performed. In an embodiment, a features from accelerated segment test (FAST) algorithm for extracting a corner using brightness information of a plurality of pixels corresponding to a circle boundary around each pixel may be used. An algorithm for calculating feature points FP according to the present disclosure is not limited thereto, and various corner detection algorithms such as Moravec algorithm, Harris algorithm, Shi & Tomasi algorithm, Wang & Brady algorithm, and SUSAN algorithm may be used. In the case of using the corner detection algorithm, feature points FP having the feature of a ‘corner’ such as an end of a toe in addition to actual vertices of the item 10 may be selected within the image IMG. FIG. illustrates that four vertices of the item 10 and five toe endpoints are calculated as feature points FP.

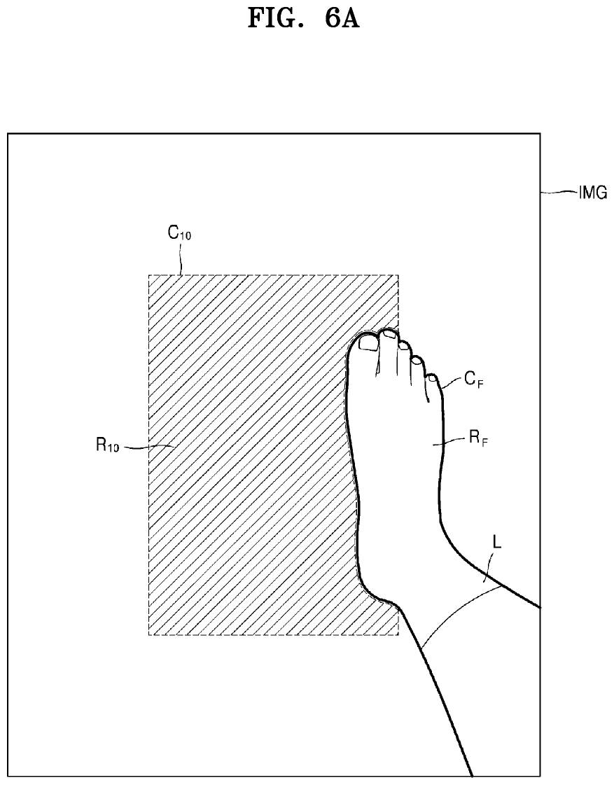

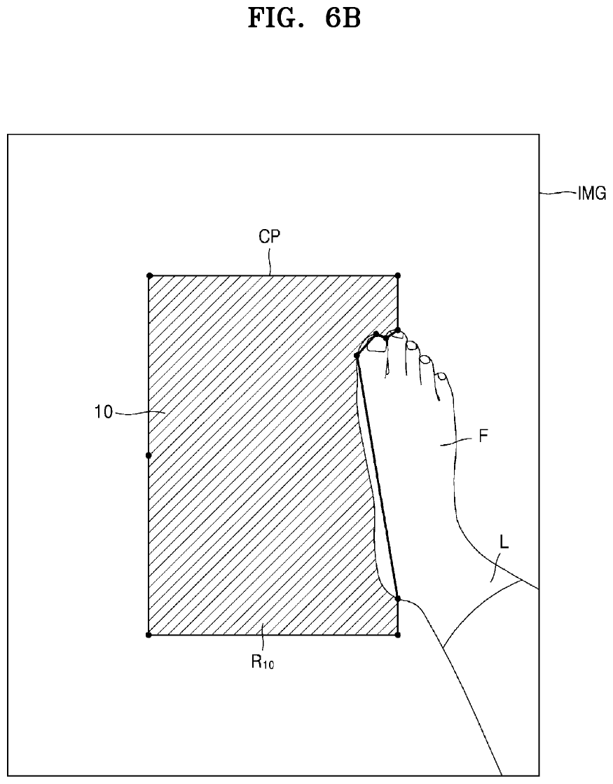

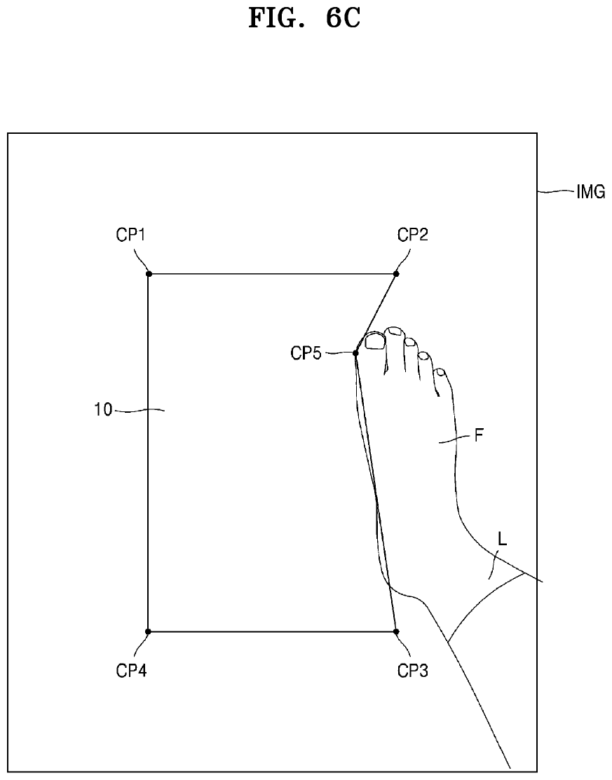

FIGS. 6A through 6C are diagrams sequentially illustrating calculating candidate points of vertices of the item 10.

Referring to FIG. 6A, extracting of a contour from the (pre-processed) image IMG may be performed. In an embodiment, Teh-Chin chain approximation algorithm may be used. However, embodiments are not limited thereto. When the extracting of the contour is performed, a contour C₁₀ of the item and a contour C_{F} of the foot may be extracted.

According to an embodiment, after the extracting of the contour is performed, selecting of a closed curve having a largest area among closed curves within the image IMG may be performed. Since, in an embodiment of the present disclosure, a A4 paper having a larger size than the foot F is generally used, it may be expected that an inner area R₁₀ of the contour C₁₀ of the item is larger than an inner area R_{F} of the contour C_{F} of the foot. A measuring apparatus may determine the closed curve having a largest area as a closed curve of the ‘item 10’.

According to an embodiment, determining whether the ratio of the inner area Rio of the closed curve of the item with respect to the entire area of the image IMG is within a preset value, may be performed. In this case, due to the characteristics of the image IMG captured centering on the item 10 and the foot F, it may be expected that the inner area R₁₀ of the item 10 in the image IMG is in a specific ratio of the entire area of the image IMG. For example, when the ratio of the inner area R₁₀ of the contour with respect to the entire area of the image IMG exceeds about 20% to about 80%, the measuring apparatus may determine that no item 10 is photographed in the image IMG or the contour C₁₀ of the item is misrecognized. In this case, the measuring apparatus may provide the user with a guide interface for re-photographing the image IMG and then may re-acquire the image IMG.

Subsequently, referring to FIG. 6B, calculating of a convex polygon CP that surrounds the contour of the item 10 may be performed. The convex polygon CP may be a set of points that surround the inner area R₁₀ of the item 10 with a smallest area among points forming the contour. Sklansky algorithm may be used to find such a convex polygon CP. However, embodiments are not limited thereto. FIG. 6B illustrates that the convex polygon CP including seven points surrounds the inner area R₁₀ of the item 10.

Subsequently, referring to FIG. 6C, calculating of candidate points CP of vertices of the item 10 through a simplification algorithm may be performed. As an algorithm for simplifying and expressing a given curve by using fewer points, Douglas-Peucker algorithm, Visvalingam algorithm, etc. may be used. However, embodiments are not limited thereto. A plurality of ‘candidate points’ may be calculated through the simplification algorithm. If the number of ‘candidate points’ is less than or equal to 3, the measuring apparatus may determine that detection of the item 10 has failed, may provide the user with an interface for guiding to re-photograph the image IMG and then may re-acquire the image IMG. FIG. 6C illustrates that five candidate points CP1 to CP5 have been detected.

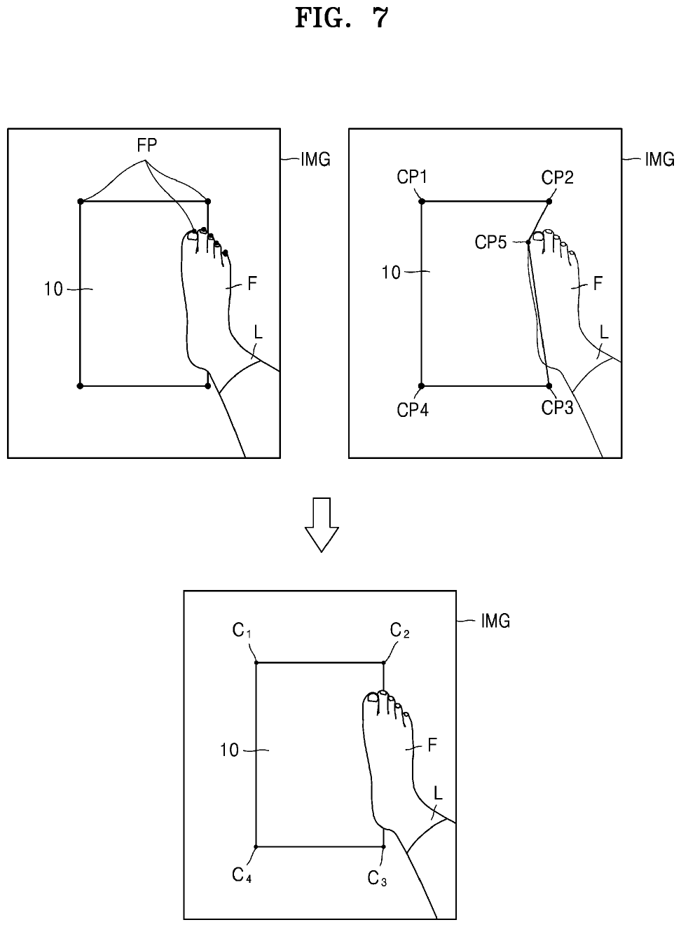

FIG. 7 is a diagram illustrating selecting of vertices C₁, C₂, C₃, and C₄ of the item 10. Previously, the ‘feature points FP’ representing the ‘corner’ and the ‘candidate points CP’ for simplifying and representing the shape of the item 10 covered by the foot F have been calculated, respectively, so that positions where the feature points FP and the candidate points CP appear in common, may be finally selected as the vertices C₁, C₂, C₃, and C₄. For example, when there are four or more feature points FP and candidate points, only points at corresponding positions are determined and selected as the vertices C₁, C₂, C₃, and C₄ through distance comparison between a plurality of feature points FP and a plurality of candidate points.

In addition, as above, a method of automatically selecting four vertices C₁, C₂, C₃, and C₄ of the item 10 through image processing has been described. However, an operation of calculating vertices may also be performed with the user's help. For example, the user may select a position near the four vertices of the item 10 by directly touching a display unit (not shown) included in the measuring apparatus or connected to the measuring apparatus. At this time, the measuring apparatus may calculate exact coordinates of the four vertices C₁, C₂, C₃, and C₄ by using a FAST algorithm for extracting a corner only near a pixel that the user touches.

After selecting of the four vertices C₁, C₂, C₃, and C₄ of the item 10 is performed, affine transformation of the image IMG according to the size of the item 10 may be performed. For example, when the item 10 is an A4 paper, the image IMG may be affine-transformed so that a square formed by the four vertices C₁, C₂, C₃, and C₄ selected from the image IMG may correspond to a rectangle having the size of 210 mm×297 mm. Thus, a size reference for acquiring size information of the foot F may be obtained.

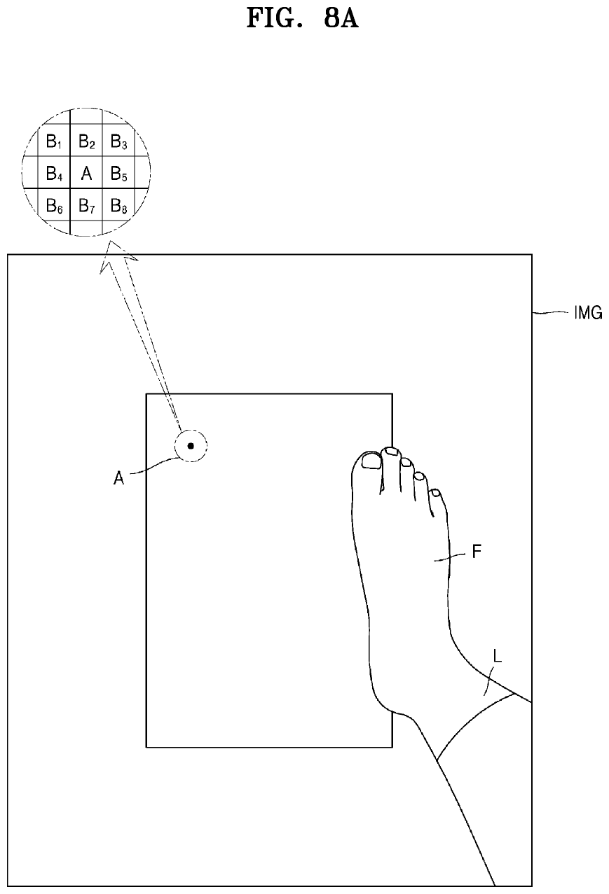

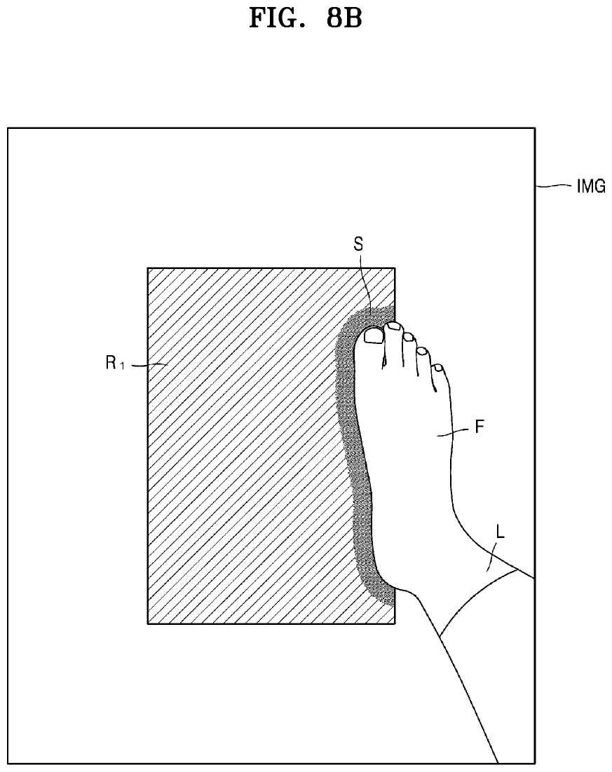

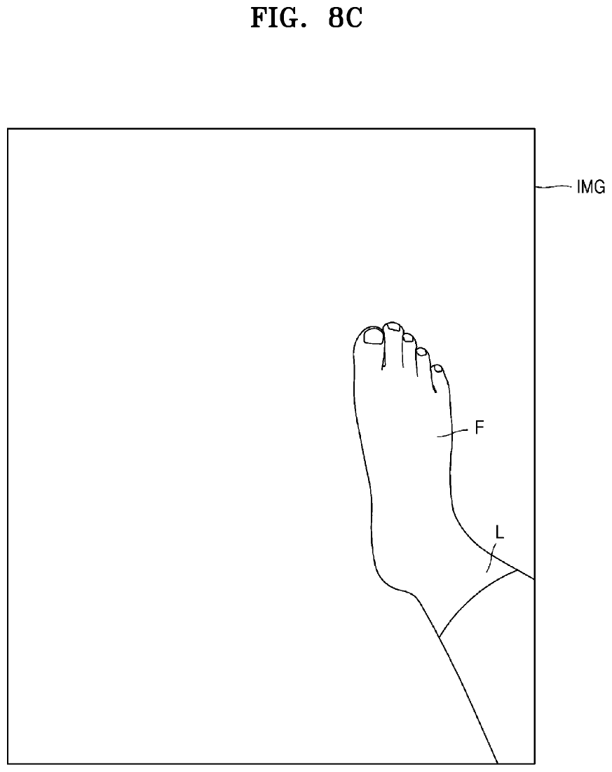

FIGS. 8A through 8C are diagrams sequentially illustrating an embodiment of calculating a region where the foot F is located in the image IMG.

Referring to FIG. 8A, one point or pixel A inside a boundary of the item 10 may be selected. The pixel A may be selected by the user or may be automatically selected as a point having a color value (CV) close to an intrinsic color of the item 10. For example, when the item 10 is an A4 paper, the pixel A may be selected as a pixel having a CV close to white among the central region of the image IMG.

Subsequently, comparing a brightness value of the selected pixel A with brightness values of pixels B₁ to B₈ around the selected pixel A may be performed. Referring to FIG. 8A, the measuring apparatus may compare a difference between grayscale values of a A-pixel and a B₁-pixel, for example. In this case, when a difference between brightness of two pixels is less than or equal to a preset threshold, the measuring apparatus may determine that the two pixels belong to the ‘same region’. That is, when the difference between brightness of the A-pixel and B₁-pixel is less than or equal to the preset threshold, it may be determined that the A-pixel and the B₁-pixel are in the ‘same region’. The brightness comparing may be repeatedly performed while the position of a pixel is changed. Finally, as shown in FIG. 8B, a first region R₁ that belongs to the ‘same region’ as the first A-pixel may be calculated. The first region R₁ may include a region where the foot F and the item 10 do not come in contact with each other, i.e., a region determined to correspond to the ‘item 10’. Since the first region R₁ is a region that does not correspond to the ‘foot F’, the first region R₁ may be removed when the region of the foot F is calculated.

In addition, when a shadow S of the foot F is photographed simultaneously in the image IMG captured by photographing the foot F, the shadow S is darkly photographed and thus is not determined to correspond to the ‘same region’ as the initial ‘A-pixel’ and may not be included in the first region R₁. In this case, according to an embodiment, histogram equalization may be performed in a portion excluding the first region R₁ from the image IMG. In this case, a boundary between an actual region of the shadow S and the region of the foot F appear clearly. Thus, the region of the shadow S and the region of the foot F may be clearly distinguished from each other using an edge detection algorithm such as Canny algorithm.

Subsequently, referring to FIG. 8C, after the first region R₁ is removed, a contour of the other region may be calculated so that a foot region R_{F} may be finally calculated and confirmed.

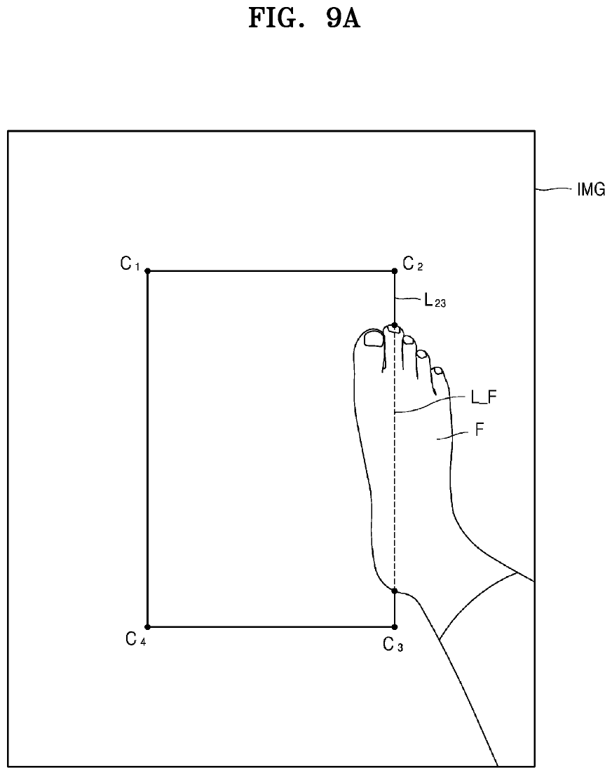

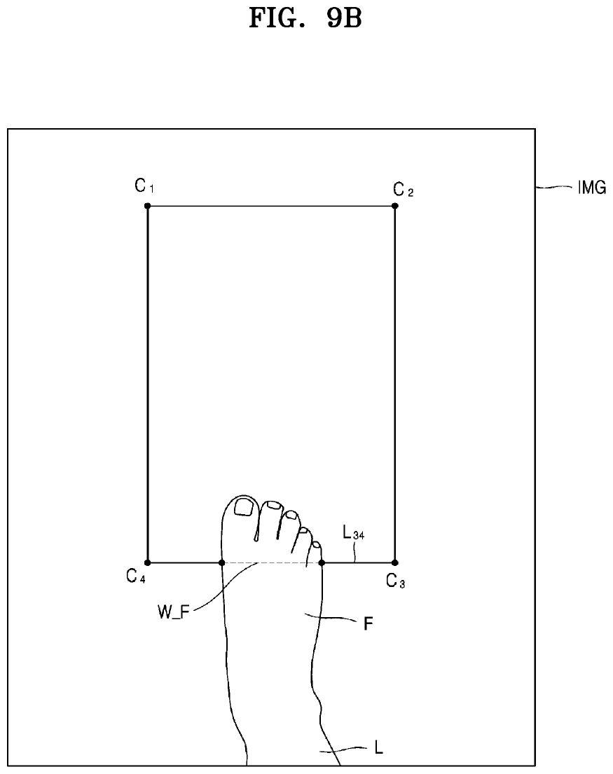

FIGS. 9A and 9B are diagrams illustrating a method for measuring the size of a foot according to an embodiment. After the region of the item 10 and the region of the foot F are calculated through image processing, the foot size such as the length of the foot F and/or the width of feet may be measured using image processing. For example, referring to FIG. 9A, a length of the foot L_F may be measured using the length of a point where the vertical edge L₂₃ of the item 10 and the region of the foot F overlap. Referring to FIG. 9B, the width of feet W_F may be measured using the length of a point where the horizontal edge L₃₄ of the item 10 and the region of the foot F overlap.

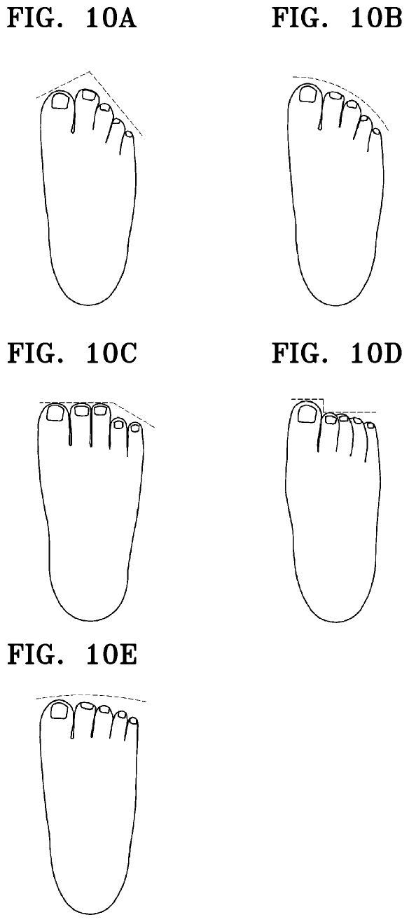

FIG. 10A through 10E are diagrams illustrating various toe shape types. According to an embodiment, calculating may include calculating a difference between relative lengths of toes from the image IMG to determine the shape type of the foot F.

When using pixel differentiation, the endpoint of the toe may be selected. In an embodiment of the present disclosure, after the image IMG is divided into regions of interest having a certain pixel, when the presence of the feature points FP within each of the regions of interest is determined and there are feature points FP, increasing the weight of the corresponding pixel and determining the presence of the feature points FP within a larger region of interest may be repeatedly performed so that a point that is likely to be the endpoint of the toe may be calculated.

After five endpoints of the toe are calculated, the shape type of the foot may be determined using a difference between positions of toes. For example, the measuring apparatus may compare the user's toe position data with foot shape type template data that has been previously stored or received from a server, to classify the user's foot type with the least error.

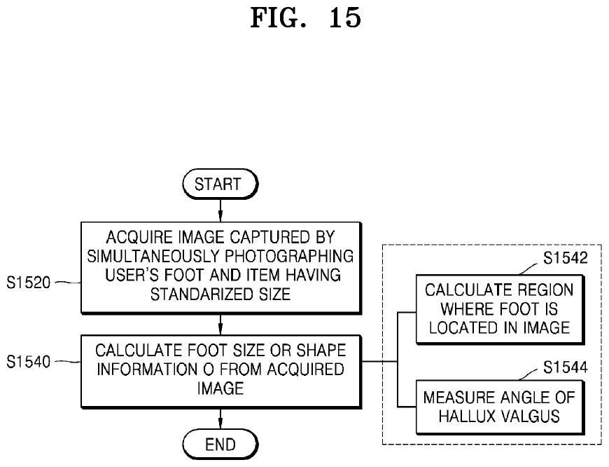

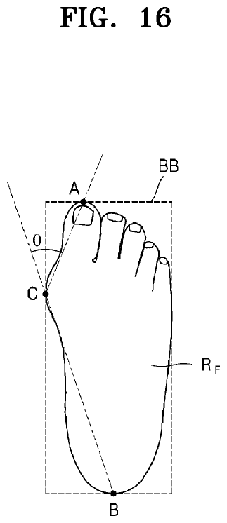

For example, when an index toe is long, as shown in FIG. 10A, the measuring apparatus may classify the user's foot as a first type. When the big toe is long and the length gradually decreases toward the little toe, as shown in FIG. 10B, the measuring apparatus may classify the user's foot as a second type. When the lengths of the big toe, the index toe and the middle toe are similar and the lengths of the ring finger toe and the little toe are short, as shown in FIG. 10C, the measuring apparatus may classify the user's foot as a third type. When the length of the big toe is long and the lengths of the other toes are similar, as shown in FIG. 10D, the measuring apparatus may classify the user's foot as a fourth type. When a difference between the lengths of the big toe and the index toe is not large, as shown in FIG. 10E, the measuring apparatus may classify the user's foot as a fifth type. The above-described foot types are exemplary, and a foot shape classification method of the present disclosure is not limited thereto. In addition, the measuring apparatus may determine whether the user has hallux valgus using the shape information of the foot, which will be described later with reference to FIGS. 15 and 16.

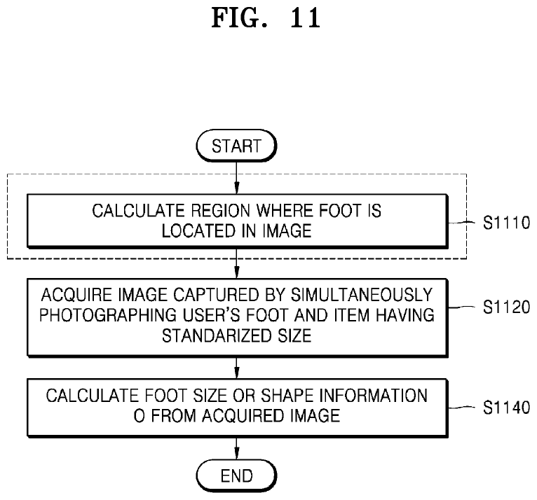

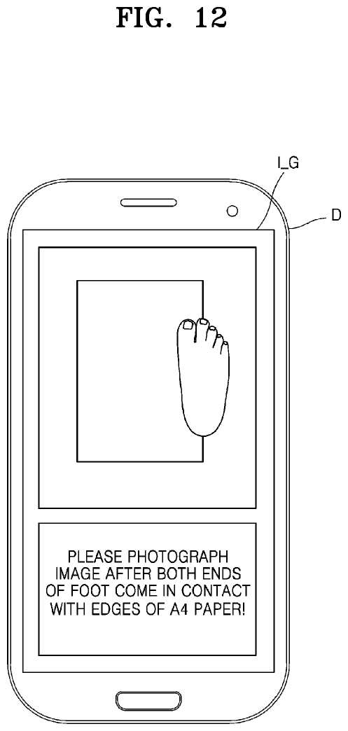

FIG. 11 is a time-sequential flowchart illustrating operations of a method for measuring foot size and shape by using image processing according to an embodiment of the present disclosure, and FIG. 12 is a diagram illustrating an interface for guiding a photographing method.

The method for measuring the foot size and shape by using image processing according to an embodiment may further include, before acquiring of the image IMG (S1120), providing a guide interface to photograph the image IMG when a part of the user's foot F comes in contact with the item 10 (S1110).

Referring to FIGS. 11 and 12, the measuring apparatus may provide/display an interface I_G for guiding a method of photographing the image IMG. Referring to FIG. 12, the measuring apparatus may provide the guide interface I_G for photographing an image “when at least a part of the user's foot comes in contact with the item” by using a photo, a text, etc. displayed on a terminal D. In detail, the measuring apparatus may provide the guide interface I_G to photograph the image so that four vertices of the item may not be covered by the user. In more detail, the measuring apparatus may provide the photographing method guide interface I_G to photograph the image by placing the foot on the edges of the A4 paper.

Subsequently, when the user photographs the image IMG according to the photographing method guided by the interface, the measuring apparatus may acquire the image IMG (S1120) and may calculate the foot size or shape information through image processing (S1140). If the user does not have an item or photographs an image ‘incorrectly’ so that the four vertices of the item 10 are not visible, the measuring apparatus that fails to detect the four vertices may provide a guide interface to re-photograph the image.

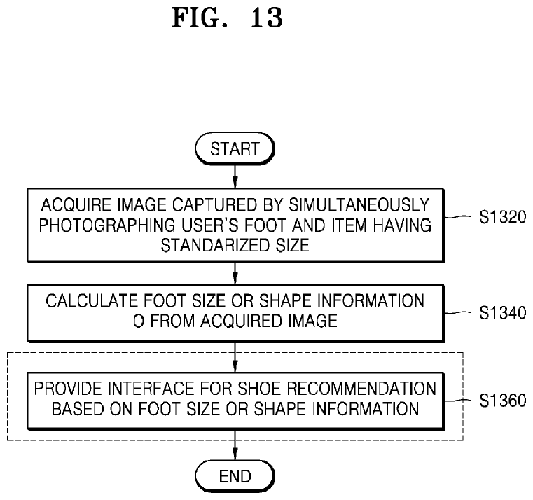

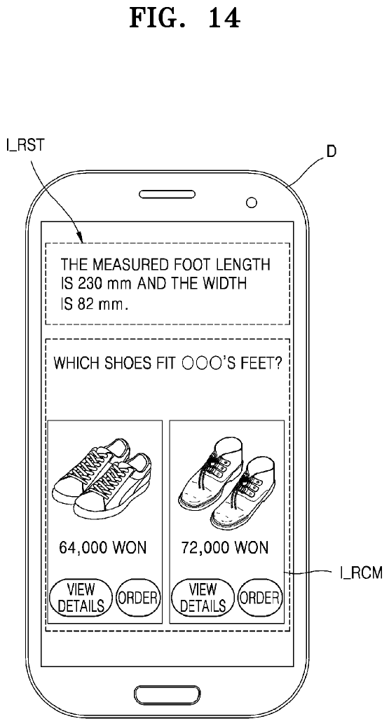

FIG. 13 is a time-sequential flowchart illustrating operations of a method for measuring foot size and shape by using image processing according to an embodiment of the present disclosure, and FIG. 14 is a diagram illustrating an interface for providing information about foot and shoes.

The method for measuring the foot size and shape by using image processing according to an embodiment may further include, after acquiring of an image IMG (S1320) and calculating of the size or shape information of the foot F (S1340) are performed, providing an interface I_RCM for shoe recommendation based on the size or shape information of the foot F (S1360).

Referring to FIG. 14, the measuring apparatus may provide an image processing result interface I_RST. In addition, the measuring apparatus may provide a shoe recommendation interface I_RCM for recommending shoes suitable for the user's foot based on the size information such as the user's foot length, the width of feet and/or shape information obtained through image processing. The shoe recommendation interface I_RCM may display information such as the photo and price of the shoes. In this case, shoes exposed to the shoe recommendation interface I_RCM may be shoes having an inner surface suitable for the user's foot size and/or shape. At this time, the measuring apparatus may provide the shoe recommendation interface I_RCM matching the user's foot size information acquired through image processing by receiving the shoe size information of various brands from a previously-stored database or server.

FIG. 15 is a time-sequential flowchart illustrating operations of a method for measuring foot size and shape by using image processing according to an embodiment of the present disclosure, and FIG. 16 is a diagram illustrating a method for measuring an angle of hallux valgus. In an embodiment of the present disclosure, the angle of hallux valgus of a patient with the symptom of hallux valgus, which is bent while the big toe joint is protruding, may be measured so that the severity of thereof may be determined.

The method for measuring the foot size and shape by using image processing according to an embodiment may include acquiring an image IMG captured by simultaneously photographing the user's foot F and the item 10 having a standardized size (S1520) and calculating size or shape information of the foot F from the image IMG (S1540). At this time, the calculating (S1540) according to an embodiment may include calculating a region where the foot F is located in the image IMG (S1542) and measuring an angle of hallux valgus of the foot from the calculated foot region (S1544).

Referring to FIGS. 15 and 16, the measuring apparatus may calculate a region R_{F} where the foot is located, and then may calculate a bounding box BB that surrounds the foot region R_{F}. For example, the bounding box BB may be a rectangle having a minimum width surrounding the foot region R_{F}. In this case, the bounding box BB and the foot region R_{F} may come in contact with each other at each of a point A where the big toe protrudes upward, an endpoint B of the heel, and a point C where the big toe joint protrudes laterally. In an embodiment of the present disclosure, an angle θ formed by a line connecting the point A and the point C and a line connecting the point B and the point C may be measured as an angle of hallux valgus. A method for measuring the angle of hallux valgus is not limited thereto.

The measuring apparatus may determine the severity of hallux valgus after measuring the angle θ of hallux valgus. The measuring apparatus may classify the severity of hallux valgus into ‘steps’ according to the size of the angle of hallux valgus. For example, the measuring apparatus may determine the severity as ‘0 step’ when the angle θ of hallux valgus is less than or equal to 12 degrees. The measuring apparatus may determine the severity as ‘1 step’ when the angle θ of hallux valgus is 12 to 20 degrees. The measuring apparatus may determine the severity as ‘step 2’ when the angle θ of hallux valgus is 20 to 30 degrees. The measuring apparatus may determine the severity as ‘step 3’ when the angle θ of hallux valgus is 30 to 50 degrees. The measuring apparatus may determine the severity as ‘step 4’ when the angle θ of hallux valgus is greater than or equal to 50 degrees. The number and numerical values of the above steps are exemplary and do not limit the present disclosure.

The measuring apparatus may provide an interface indicating severity information of hallux valgus. For example, the measuring apparatus may provide an interface displaying information ‘this step is an initial step of a hallux valgus symptom and thus please pay attention to shoe selection’, when the measured severity is at step 1. In addition, the measuring apparatus may provide an interface for recommending shoes that are comfortable for hallux valgus patients or a hospital specializing in hallux valgus or displaying lifestyle information required for hallux valgus patients, based on the angle θ of hallux valgus or information about the severity of hallux valgus.

The method according to the above-described embodiment may be implemented in the form of programs and application program instructions that can be executed through various computer means, and recorded in a computer-readable medium. The computer-readable medium may include program instructions, data files, data structures, and the like alone or in combination. The program instructions recorded on the medium may be specially designed and configured for the embodiment, or may be known and usable to those skilled in computer software. Examples of computer-readable recording media include magnetic media such as hard disks, floppy disks, and magnetic tapes, optical media such as CD-ROMs and DVDs, magneto-optical media such as floptical disks, and a hardware device specially configured to store and execute program instructions such as ROMs, RAMs, flash memory, and the like. Examples of the program instructions include not only machine language codes such as those produced by a compiler, but also high-level language codes that can be executed by a computer using an interpreter or the like. The above-described hardware device may be configured to operate as one or more software modules to perform the operation of the embodiment, and vice versa.

The above-described method of the present disclosure can be executed through an application program stored in a computer-readable recording medium in combination with hardware such as a mobile device such as a smartphone and a tablet. For example, the user photographs the foot F and the item 10 by using a camera built into the smartphone and uploads it to the application program. The application program may analyze the image uploaded/input by the user, measure the size and shape of the user's foot, and recommend shoes matching the user's foot size information.

In the method for measuring foot size and shape by using image processing according to an embodiment of the present disclosure, the user can measure the foot size and/or shape automatically/semi-automatically through an image captured by a user through photographing. Thus, the user can not only easily know information about his/her own foot size conveniently, but also can receive information about shoes that do fit his/her own feet.

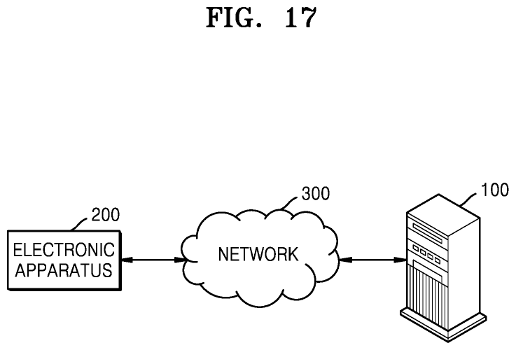

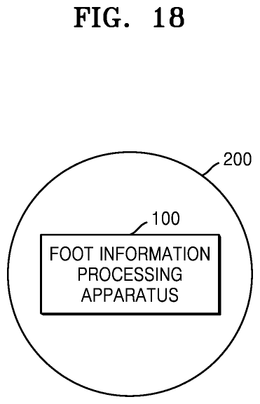

FIG. 17 is a diagram of an example of a foot information processing environment including a foot information processing apparatus, an electronic apparatus, and a network connecting them to each other, according to an embodiment, and FIG. 18 is a diagram of an example of a foot information processing environment according to another embodiment. Referring to FIGS. 17 and 18, the foot information processing environment may include a foot information processing apparatus 100, an electronic apparatus 200, and a network 300.

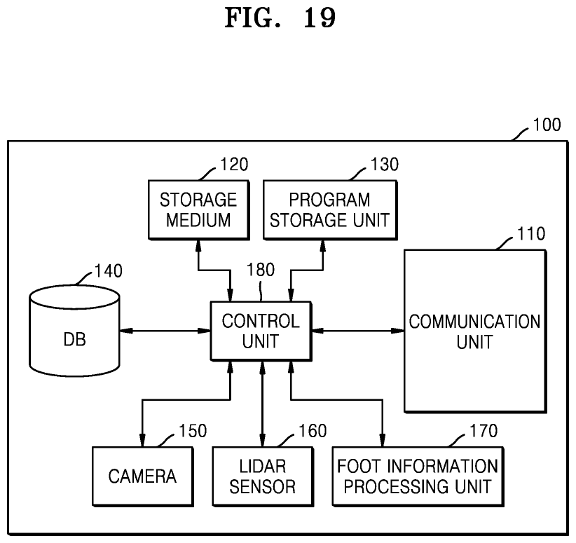

The foot information processing apparatus 100 may acquire various pieces of information based on a plurality of foot images taken from various angles. The foot information processing apparatus 100 may acquire two-dimensional (2D) red-green-blue (RGB) information from a plurality of foot images captured by a camera 150 (see FIG. 19). The foot information processing apparatus 100 may acquire pieces of three-dimensional (3D) point cloud data (a 3D point cloud set) based on information about a distance to a foot, which is measured by a light-detection-and-ranging (LiDAR) sensor 160 (see FIG. 19). The foot information processing apparatus 100 may acquire information about a height from the ground to the camera, which is measured by a gravitational acceleration sensor (not shown).

The foot information processing apparatus 100 may generate an initial 3D model of the foot by combining the RGB information with the pieces of 3D point cloud data based on the information about the distance to the foot. The foot information processing apparatus 100 may generate an intermediate 3D model by removing the ground and noise from the initial 3D model. The foot information processing apparatus 100 may generate a final 3D model with a normal foot shape by restoring a portion of the foot that has been removed along with the ground when generating the intermediate 3D model.

The foot information processing apparatus 100 may calculate foot-related information from the final 3D model and recommend a type and size of shoes. Here, the foot-related information may include length information of the foot, width information of the foot, circumference information of the foot, thickness information of the foot, shape information of the foot, and instep height information of the foot, which are calculated from the final 3D model.

The foot information processing apparatus 100 may recommend a type and size of shoes by using an artificial intelligence algorithm. Here, artificial intelligence (AI) is a field of computer engineering and information technology for researching a method for allowing computers to do thinking, learning, self-development or the like that can be done by human intelligence, and may refer to a process of causing a computer to imitate human intelligent behavior.

In addition, AI does not exist on its own, but is rather directly or indirectly connected with other fields in computer science. In recent years, there have been extensive attempts to use AI for problem solving in the field of information technology.

Machine learning is an application of AI that gives computers the ability to automatically learn and improve from experience without explicit programs. In detail, machine learning is a technique for researching and building a system that performs learning based on empirical data, performs predictions, and improves its own performance, and algorithms therefor. The algorithms in machine learning may take a way of building specific models to derive predictions or decisions based on input data, rather than performing strictly defined static program instructions.

Both unsupervised learning and supervised learning may be used as machine learning methods for such AI networks. In addition, in addition, deep learning technology, which is a subfield of machine learning, may enable multi-step, deep-level learning based on data. Deep learning may refer to a set of machine learning algorithms for extracting key data from a plurality of pieces of data as the number of steps increases.

The foot information processing apparatus 100 may recommend shoes corresponding to the foot-related information by using a deep neural network model that is pretrained to recommend a type and size of shoes by using foot-related information as an input. Here, the deep neural network model may be a model trained in a supervised learning manner by using training data including foot-related information as inputs, and types and sizes of shoes as labels.

In the present embodiment, the foot information processing apparatus 100 may be implemented as an independent server as illustrated in FIG. 17, or a foot information processing function provided by the foot information processing apparatus 100 may be implemented as an application to be installed in the electronic apparatus 200 as illustrated in FIG. 18.

The electronic apparatus 200 may receive a foot information processing service by accessing a foot information processing application and/or a foot information processing site provided by the foot information processing apparatus 100. The electronic apparatus 200 may be an apparatus capable of performing a function of a computing device (not shown) and equipped with a camera, and may include, for example, a desktop computer, a smartphone, a tablet personal computer (PC), a notebook computer, a camcorder, a webcam, etc.

The network 300 may serve to connect the foot information processing apparatus 100 to the electronic apparatus 200. The network 300 may include, for example, a wired network such as a local area network (LAN), a wide area network (WAN), a metropolitan area network (MAN), or an integrated services digital network (ISDN), or a wireless network such as a wireless LAN (WLAN), code-division multiple access (CDMA), Bluetooth, or satellite communication, but the scope of the present disclosure is not limited thereto. In addition, the network 300 may transmit and receive information by using short-range communication and/or long-range communication. Here, the short-range communication may include Bluetooth, radio-frequency identification (RFID), Infrared Data Association (IrDA), ultra-wideband (UWB), ZigBee, and wireless fidelity (Wi-Fi), and the long-range communication may include code-division multiple access (CDMA), frequency-division multiple access (FDMA), time-division multiple access (TDMA), orthogonal FDMA (OFDMA), and single-carrier FDMA (SC-FDMA).

The network 300 may include connection of network elements, such as hubs, bridges, routers, or switches. The network 300 may include one or more connected networks, for example, a multi-network environment, including a public network, such as the Internet, and a private network, such as a secure corporate private network. Access to the network 300 may be provided through one or more wired or wireless access networks. Furthermore, the network 300 may support an Internet-of-Things (IoT) network and/or 5^{th} Generation (5G) communication for exchanging and processing information between distributed components such as objects.

In the present embodiment, the plurality of foot images captured by the electronic apparatus 200 may be stored in a Universal Serial Bus (USB) (universal serial bus) memory card (not shown), which is one of flash storage media included in a storage medium 120 (see FIG. 19). When foot information processing is necessary thereafter, the USB memory card may be connected to the electronic apparatus 200, such that a single 2D image may be transferred from the USB memory card to the electronic apparatus 200 and then transmitted to the foot information processing apparatus 100 through the network 300.

FIG. 19 is a block diagram schematically illustrating a configuration of a foot information processing apparatus according to an embodiment. In the following description, redundant descriptions provided above with reference to FIGS. 1 and 2 will be omitted. Referring to FIG. 18, the foot information processing apparatus 100 may include a communication unit 110, the storage medium 120, a program storage unit 130, a database 140, the camera 150, the LiDAR sensor 160, a foot information processing unit 170, and a control unit 180.

The communication unit 110 may provide a communication interface necessary to provide signals transmitted and received between the foot information processing apparatus 100 and the electronic apparatus 200 in the form of packet data in conjunction with the network 300. Furthermore, the communication unit 110 may serve to receive a certain information request signal from the electronic apparatus 200, and transmit information processed by the foot information processing unit 170 to the electronic apparatus 200. Here, a communication network is a medium that serves to connect the foot information processing apparatus 100 to the electronic apparatus 200, and may include a path for providing an access path to allow the electronic apparatus 200 having accessed the foot information processing apparatus 100 to transmit and receive information. In addition, the communication unit 110 may be a device including hardware and software necessary for transmitting and receiving signals, such as control signals or data signals, through wired/wireless connection with other network devices.

The storage medium 120 performs a function of temporarily or permanently storing data processed by the control unit 180. Here, the storage medium 120 may include a magnetic storage medium or a flash storage medium, but the scope of the present disclosure is not limited thereto.

The program storage unit 130 is equipped with control software for performing an operation of acquiring 2D RGB information based on a plurality of foot images taken from various angles, an operation of acquiring pieces of 3D point cloud data based on information about a distance to a foot, which is measured by the LiDAR sensor 160 (see FIG. 19), an operation of obtaining information about a height from the ground to a camera, which is measured by a gravitational acceleration sensor, an operation of generating an initial 3D model of the foot by combining the RGB information with the pieces of 3D point cloud data based on the information about the distance to the foot, an operation of generating an intermediate 3D model by removing the ground and noise from the initial 3D model, an operation of generating a final 3D model with a normal foot shape by restoring a portion of the foot that has been removed along with the ground when generating the intermediate 3D model, and an operation of calculating foot-related information from the final 3D model and recommending a type and size of shoes.

The database 140 may include a management database for storing foot images collected from the electronic apparatus 200, 2D RGB information obtained based on a foot image, information about a distance to a foot, information about a height from the ground to the camera, an initial 3D model, an intermediate 3D model, and a final 3D model of the foot, foot-related information calculated from the final 3D model, an artificial intelligence algorithm for recommending a type and size of shoes, an algorithm for removing the ground by using the intermediate 3D model, an algorithm for removing noise from the intermediate 3D model, a Poisson surface reconstruction algorithm for reconstructing the intermediate 3D model, etc.

The database 140 may include a user database for storing information of a user to be provided with a foot information processing service. Here, the information of the user may include basic information such as the user's name, affiliation, personal information, gender, age, contact information, email, address, or photo, information about user authentication (login), such as an identifier (ID) (or an e-mail) or a password, and access-related information, such as a country of access, a location of access, information about a device used for access, or a network environment of access.

In addition, the user database may store the user's unique information, information and/or a category history provided to the user who accessed the foot information processing application or the foot information processing site, information about environment settings by the user, information about resources used by the user, billing and payment information with respect to the user's resource usage.

The camera 150 may include an RGB camera mounted on one side of the foot information processing apparatus 100 to generate a plurality of foot images by photographing a foot from various angles (e.g., 360 degrees). Here, each foot image may refer to 2D image information containing color information similar to human visual perception, that is, RGB information.

The RGB camera may basically recognize the shape and color of an object similarly to human vision. However, the RGB camera expresses visible light reflected from an object (e.g., a foot) as image information, and thus has the disadvantage of being vulnerable to external environmental factors such as lighting, weather, or cutting of an object. In addition, there are many difficulties in acquiring accurate 3D distance information of an object (e.g., a foot) detected through the RGB camera. Therefore, recently, in order to improve the performance of object detection, the LiDAR sensor 160 may be used together with the camera 150 to compensate for the limitations.

The LiDAR sensor 160 may emit a laser and represent a signal reflected from the foot within a measurement range, as pieces of 3D point cloud data. The LiDAR sensor 160 measures a reflected signal from a laser derived from the LiDAR sensor 160 itself, and thus may have the advantage of being robust to external environmental factors, unlike the camera 150 that measures visible light. In addition, it is possible to accurately measure the distance to the foot, including reflectance information according to surface properties, and distance information according to a time of reflection. However, because only reflected laser signals are measured, only environmental information contained in a reflection area is represented, accordingly, the resolution of data represented by pieces of 3D point cloud data is significantly low, less than 10% of image information, and thus, there may be limitations in expressing all information in a real environment.

The foot information processing unit 170 may acquire 2D RGB information from a plurality of foot images captured by the camera 150, pieces of 3D point cloud data based on information about a distance to a foot, which is measured by the LiDAR sensor 160, information about a height from the ground to the camera, which is measured by a gravitational acceleration sensor (not shown).

The foot information processing unit 170 may generate an initial 3D model of the foot by combining the RGB information with the pieces of 3D point cloud data based on the information about the distance to the foot. The foot information processing unit 170 may generate an intermediate 3D model by removing the ground and noise from the initial 3D model. The foot information processing unit 170 may generate a final 3D model with a normal foot shape by restoring a portion of the foot that has been removed along with the ground when generating the intermediate 3D model.

The foot information processing unit 170 may calculate foot-related information from the final 3D model and recommend a type and size of shoes. Here, the foot-related information may include length information of the foot, width information of the foot, circumference information of the foot, thickness information of the foot, shape information of the foot, and instep height information of the foot, which are calculated from the final 3D model. The foot information processing unit 170 may recommend a type and size of shoes by using an artificial intelligence algorithm.

The control unit 180 is a central processing unit, and may control the overall operation of the foot information processing apparatus 100 by executing control software stored in the program storage unit 130. The control unit 180 may include all types of devices capable of processing data, such as a processor. Here, the ‘processor’ may refer to a hardware-embedded data processing device having a physically structured circuitry to perform functions represented by code or instructions included in a program. Examples of the hardware-embedded data processing device may include a processing device, such as a microprocessor, a central processing unit (CPU), a processor core, a multiprocessor, an application-specific integrated circuit (ASIC), and a field-programmable gate array (FPGA), but the scope of the present disclosure is not limited thereto.

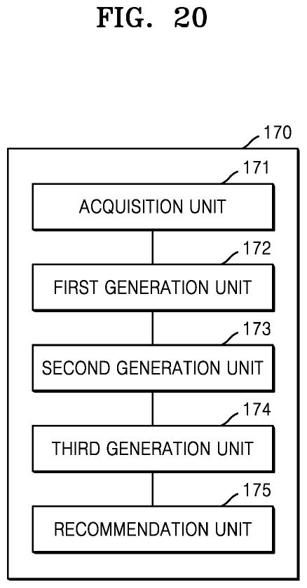

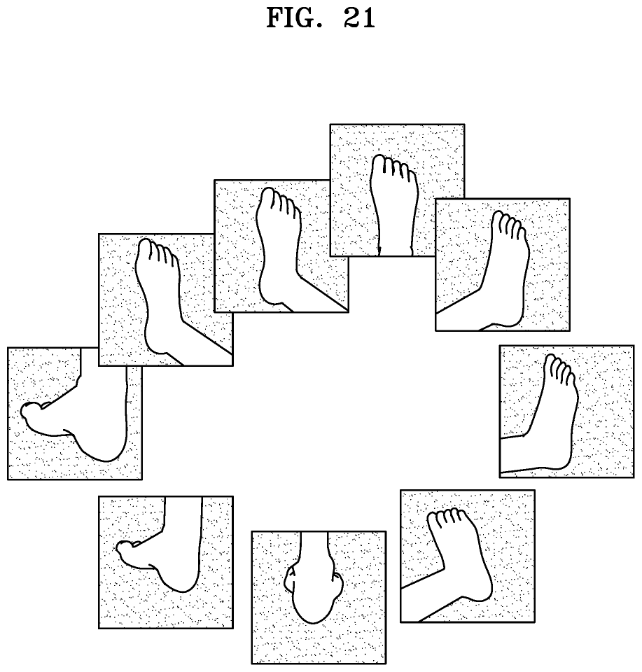

FIG. 20 is a block diagram for schematically describing a configuration of a foot information processing unit according to an embodiment, and FIGS. 21 to 24 are diagrams of examples for describing foot information processing according to an embodiment. In the following description, redundant descriptions provided above with reference to FIGS. 17 to 19 will be omitted. Referring FIGS. 20 to 24, the foot information processing unit 170 may include an acquisition unit 171, a first generation unit 172, a second generation unit 173, a third generation unit 174, and a recommendation unit 175.

The acquisition unit 171 may acquire 2D RGB information from a plurality of foot images taken from various angles (e.g., 360 degrees) by using the camera 150. FIG. 21 illustrates a plurality of foot images taken from various angles centered on a foot by using the camera 150.

When capturing the plurality of foot images, the acquisition unit 171 may acquire pieces of 3D point cloud data (a 3D point cloud set) based on information about a distance to the foot, which is measured by the LiDAR sensor 160. Here, the point cloud data refers to one of methods of expressing 3D data, i.e., a method of expressing a point in three dimensions, and may be in a vector form that may include both position coordinates and a color. For example, the point cloud data may be expressed as (x, y, z, R, G, B). A significantly large amount of color and position data constitutes a spatial configuration of a point cloud, and as its density increases, the point cloud becomes more detailed with respect to data and thus may be more meaningful as a 3D model.

The acquisition unit 171 may acquire information about a height from the ground to the camera 150, which is measured by the gravitational acceleration sensor.

In the present embodiment, in a case in which the foot information processing apparatus 100 is mounted on a smartphone serving as the electronic apparatus 200, the foot information processing apparatus 100 may generate and store a plurality of foot images by capturing a foot from various angles (e.g., 360 degrees) by using a camera provided on the smartphone, calculate and store position coordinates of the smartphone when capturing an image of the foot, calculate and store pieces of 3D point cloud data based on information about a distance from the smartphone to the foot by using a LiDAR sensor provided in the smartphone, and calculate and store information about a height from the ground to the smartphone by using a gravitational acceleration sensor provided in the smartphone.

Furthermore, the acquisition unit 171 may detect the foot as a central object from the plurality of foot images. That is, the acquisition unit 171 may identify where the foot is in an image captured by the camera 150. The acquisition unit 171 may detect the foot from the image by using an anchor-based object detection algorithm. In anchor-based object detection, a foot as an object may be detected by arranging an anchor in each cell constituting a feature map of an object detection network and performing learning of objectness, class scores, object locations, and shapes.

In addition, the acquisition unit 171 may recognize a foot as an object by using the Augmented Reality Kit (ARKit) algorithm that measures the size of an object, and acquire a result of analysis of surrounding 3D environments, including a result of measurement of the foot.

The first generation unit 172 may generate an initial 3D model of the foot by combining the RGB information acquired from the plurality of foot images with the pieces of 3D point cloud data.

The second generation unit 173 may generate an intermediate 3D model by removing the ground and noise from the initial 3D model.

The second generation unit 173 may detect one or more planes by using the pieces of 3D point cloud data included in the initial 3D model, and estimate and remove one of the one or more planes as the ground based on the information about the height from the ground to the camera, which is obtained when capturing the plurality of foot images.

The second generation unit 73 may use a random sample consensus (RANSAC) algorithm for detecting the one or more planes. The RANSAC algorithm may randomly select minimum points (three minimum points for a plane) from the pieces of 3D point cloud data to estimate a plane, and when the number of points supporting the plane is greater than or equal to a preset threshold, detect the points as a plane.

In another embodiment, the second generation unit 173 may detect one or more planes by using a normal vector. A normal vector corresponding to each pixel may be generated by comparing distance information, and pixels with similar normals may be grouped into one and detected as one plane. The well-known region growing algorithm may be applied to group the pixels, and when normal vectors of pixels differ from each other by a preset angle or greater, or depths of pixels differ from each other by more than a preset distance or greater, the pixels may be assumed to be different planes so as to prevent a region from being expanded.

The second generation unit 173 may estimate, as the ground, one of the detected one or more planes by using the information about the height from the ground to the camera 150, which is acquired when capturing the plurality of foot images, and remove it.

For example, when there are three planes detected by the second generation unit 173 using the initial 3D model of the foot, and the information about the height from the ground to the camera 150 is 50 cm, the second generation unit 173 may estimate, as the ground, a plane located below the information about the height (50 cm) in the initial 3D model of the foot, and remove it.

In the present embodiment, photographing conditions may be set and may include a condition that the foot be photographed with the sole being in contact with the ground. Thus, when the second generation unit 173 determines a plane from the initial 3D model of the foot and removes the ground, the sole may also be removed.

In addition, the initial 3D model of the foot may contain noise, and such noise may occur when capturing the plurality of foot images, or when generating the initial 3D model of the foot.

For example, in a case in which the foot information processing apparatus 100 is mounted on a smartphone serving as the electronic apparatus 200, when photographing a foot by using the smartphone, noise may be generated as the foot moves or the smartphone moves. In addition, noise may occur when combining the RGB information with the pieces of 3D point cloud data to generate create the initial 3D model of the foot. Because a general image synthesis method is used, noise that may occur during general image synthesis may be contained.

The second generation unit 173 may remove noise generated when capturing the plurality of foot images or noise generated when generating the initial 3D model of the foot, by using cluster analysis. In an embodiment, the second generation unit 173 may remove noise by using a density-based spatial clustering of applications with noise (DBSCAN) algorithm.

The DBSCAN algorithm is a technique that regards a dense region as one cluster, and when the region is connected to another dense region, performs cluster analysis while expanding the cluster. The most important decision in the DBSCAN algorithm may be how to define a dense region. To define a dense region, two concepts may be used: a designated distance e and a minimum number n of pieces of required data within the designated distance e. The designated distance e refers to a distance to be searched with respect to a certain data point, and the minimum number n of pieces of data may refer to the number of pieces of required data within the designated distance e with respect to corresponding data. The DBSCAN algorithm has the advantage that it does not require selecting the number of clusters, and it classifies noise points, thus is robust to noise, and may also be used to search for an outlier. In addition, the DBSCAN algorithm may be applied to data with a complex or geometric shape, by connecting dense regions to generate a cluster.

In order to remove noise, the second generation unit 173 may calculate distances between the pieces of 3D point cloud data included in the initial 3D model of the foot, and calculate the density of the pieces of 3D point cloud data. The second generation unit 173 may extract a region where the calculated density is greater than or equal to a reference value and where a cluster is formed, from among the pieces of 3D point cloud data. The second generation unit 173 may collect regions similar to a general foot shape model, from the extracted region, estimate the regions as a foot, determine, as noise, a portion that is different from the foot shape model, and remove the noise.

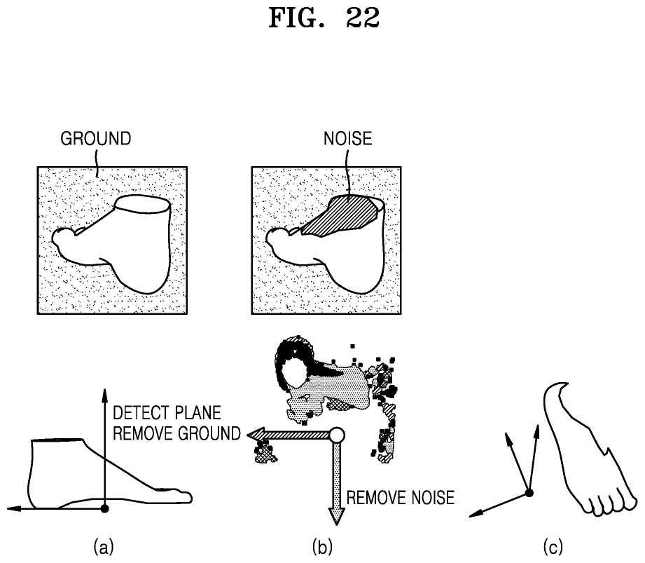

FIG. 22 illustrate a process in which the second generation unit 173 generates an intermediate 3D model. FIG. 22(a) illustrates that a plane is detected from an initial 3D model of a foot, the plane corresponding to the ground is removed by using information about a height from the ground to the camera 150, and the sole is also removed when removing the plane corresponding to the ground. FIG. 22(b) illustrates removal of noise that has been generated when capturing an image of the foot or when generating an initial 3D model of the foot. FIG. 22(c) illustrates an intermediate 3D model of the foot generated through removal of the ground and noise.

The third generation unit 174 may generate a final 3D model with a normal foot shape by restoring a portion of the foot that has been removed along with the ground when generating the intermediate 3D model.

The third generation unit 174 may detect an outline forming the surface of the foot, from the pieces of 3D point cloud data having a preset height from the ground, among the pieces of 3D point cloud data included in the intermediate 3D model. The third generation unit 174 may generate a sole portion by vertically projecting, onto the ground, the pieces of 3D point cloud data having the preset height from the ground and corresponding to the outline. The third generation unit 174 may restore the sole portion generated by vertically projecting the pieces of 3D point cloud data onto the ground, as a portion of the foot that has been removed along with the ground.

The third generation unit 174 may generate a final 3D model by interpolating the intermediate 3D model in which the sole portion is restored. Here, the interpolating may include smoothing the 3D model and filling in empty portions.

In an embodiment, the third generation unit 174 may perform a interpolation process by aligning normal vectors by using a virtual camera position (the position where a foot image was actually captured). In a case in which the foot information processing apparatus 100 is mounted on a smartphone serving as the electronic apparatus 200, the smartphone is moved while capturing foot images, and thus, a plurality of captured foot images may not correspond to each other in position. Thus, when generating a 3D model by using foot images captured by using a smartphone, the 3D model may contain some rough portions. An interpolation process may be performed to correct the rough portions generated as described above, by aligning normal vectors of the rough portions.

In another embodiment, the third generation unit 174 may perform an interpolation process by using a Poisson surface reconstruction algorithm. The sole portion generated by vertically projecting the pieces of 3D point cloud data of the outline onto the ground may not be as smooth as the actual shape of the foot, and in this case, an interpolation process may be performed to correct the 3D model to be smooth by using the Poisson surface reconstruction algorithm that predicts the surface based on values of surrounding regions.

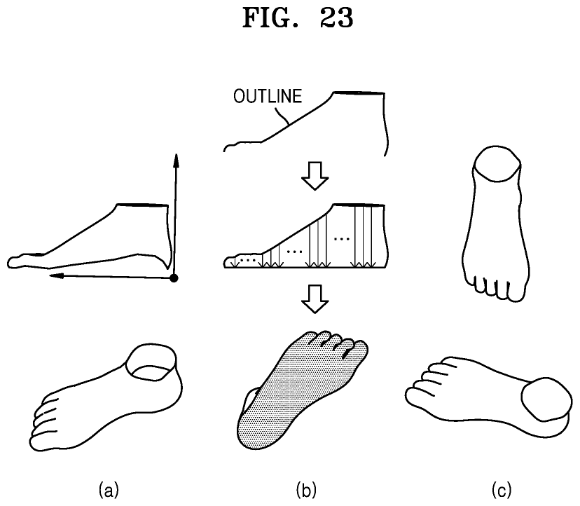

FIG. 23 illustrate a process in which the third generation unit 174 generates a final 3D model. FIG. 23(a) illustrates an intermediate 3D model from which a sole portion is removed. FIG. 23(b) illustrates an example in which a sole portion is generated by detecting an outline forming the surface of a foot, from pieces of 3D point cloud data, and vertically projecting the pieces of 3D point cloud data corresponding to the outline onto the ground. FIG. 23(c) illustrates a final 3D model generated by restoring the sole portion and performing an interpolation process.

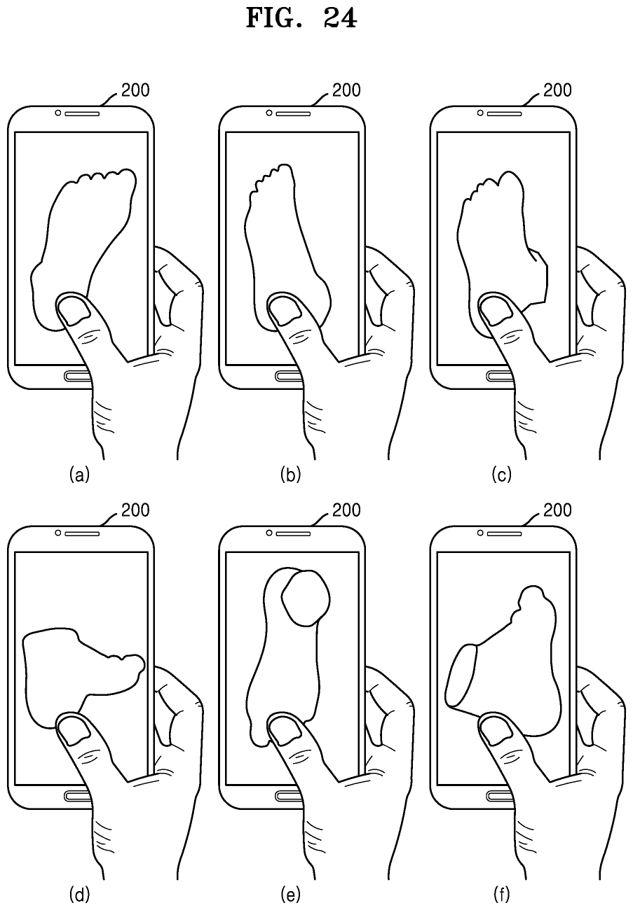

FIG. 24 illustrates an example in which, the foot information processing apparatus 100, which is mounted on a smartphone serving as the electronic apparatus 200, outputs a final 3D model generated to the smartphone, and the final 3D model is viewed from various angles through a user's touch-and-drag manipulation. The user of the smartphone may receive a recommendation on a type and size of shoes that are suitable for the user, by using a final 3D model generated by capturing images of the user's foot.

The recommendation unit 175 may calculate foot-related information from the final 3D model and recommend a type and size of shoes. Here, the foot-related information may include length information of the foot, width information of the foot, circumference information of the foot, thickness information of the foot, shape information of the foot, and instep height information of the foot.

The recommendation unit 175 may recommend a type and size of shoes corresponding to the foot-related information by using a deep neural network model that is pretrained to recommend a type and size of shoes by using foot-related information as an input. Here, the deep neural network model may be a model trained in a supervised learning manner by using training data including foot-related information as inputs, and types and sizes of shoes as labels.

The recommendation unit 175 may train an initially set deep neural network model by using labeled training data in a supervised learning manner. Here, the initially set deep neural network model is an initial model designed to be configured as a model capable of recommending a type and size of shoes, and its parameter values are set to arbitrary initial values. The parameter values are optimized while the initial model is trained through the above-described training data, and thus, the model is completely trained into a shoe recommendation model capable of recommending a type and size of shoes.

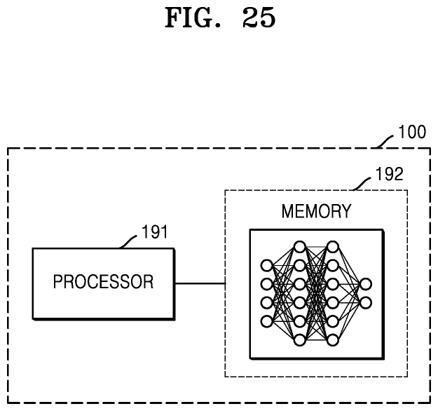

FIG. 25 is a block diagram schematically illustrating a configuration of a foot information processing apparatus according to another embodiment. In the following description, redundant descriptions provided above with reference to FIGS. 17 to 24 will be omitted. Referring to FIG. 25, in another embodiment, a foot information processing apparatus may include a processor 191 and a memory 192.

In the present embodiment, the processor 191 may process functions performed by the communication unit 110, the storage medium 120, the program storage unit 130, the database 140, the camera 150, the LiDAR sensor 160, the foot information processing unit 170, and the control unit 180, which are illustrated in FIG. 19, and the foot information processing unit 170 including the acquisition unit 171, the first generation unit 172, the second generation unit 173, the third generation unit 174, and the recommendation unit 175, which are illustrated in FIG. 20.

The processor 191 may control the overall operation of the foot information processing apparatus 100. Here, the ‘processor’ may refer to a hardware-embedded data processing device having a physically structured circuitry to perform functions represented by code or instructions included in a program. Examples of the hardware-embedded data processing device may include a processing device, such as a microprocessor, a CPU, a processor core, a multiprocessor, an ASIC, and an FPGA, but the scope of the present disclosure is not limited thereto.

The memory 192 may be operatively connected to the processor 191 and may store at least one piece of code associated with an operation performed by the processor 191.

In addition, the memory 192 may perform a function of temporarily or permanently storing data processed by the processor 191, and, in an embodiment, may store data stored in the database 140 of FIG. 19. Here, the memory 192 may include a magnetic storage medium or a flash storage medium, but the scope of the present disclosure is not limited thereto. The memory 192 may include an internal memory and/or an external memory, and may include a volatile memory such as a dynamic random-access memory (DRAM), a static random-access memory (SRAM), or a synchronous DRAM (SDRAM), a nonvolatile memory such as a one-time programmable read-only memory (OTPROM), a programmable read-only memory (PROM), an erasable programmable read-only memory (EPROM), an electrically erasable programmable read-only memory (EEPROM), a mask read-only memory (ROM), a flash ROM, a NAND flash memory, or a NOR flash memory, a flash drive such as a solid-state drive (SSD), a compact flash (CF) card, a Secure Digital (SD) card, a Micro-SD card, a Mini-SD card, an eXtreme Digital (XD) card, or a memory stick, or a storage device such as a hard disk drive (HDD).

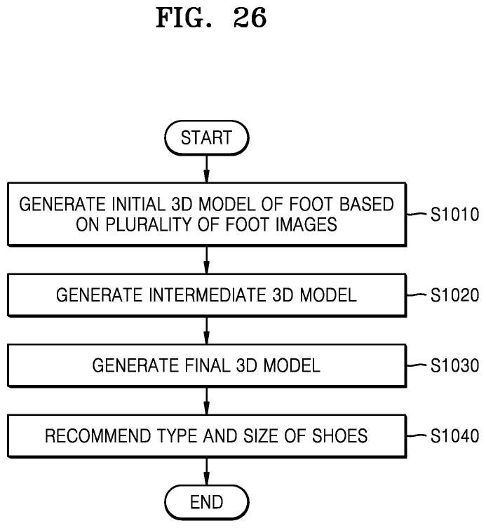

FIG. 26 is a flowchart for describing a foot information processing method according to an embodiment. In the following description, redundant descriptions provided above with reference to FIGS. 17 to 25 will be omitted.

Referring to FIG. 26, in operation S1010, the foot information processing apparatus 100 may generate an initial 3D model of a foot based on a plurality of foot images taken from various angles. The foot information processing apparatus 100 may acquire RGB information from the plurality of foot images that are captured while moving the camera 360 degrees around the foot. When capturing the plurality of foot images, the foot information processing apparatus 100 may acquire pieces of 3D point cloud data (a 3D point cloud set) based on information about a distance to the foot, which is measured by a LiDAR sensor. The foot information processing apparatus 100 may generate an initial 3D model of the foot by combining the RGB information with the pieces of 3D point cloud data based on the information about the distance to the foot.

In operation S1020, the foot information processing unit 170 may generate an intermediate 3D model by removing the ground and noise from the initial 3D model. The foot information processing apparatus 100 may detect one or more planes by using the pieces of 3D point cloud data included in the initial 3D model, estimate and remove one of the one or more planes as the ground based on the information about the height from the ground to the camera, which is obtained when capturing the plurality of foot images. The foot information processing apparatus 100 may remove noise generated when capturing the plurality of foot images or noise generated when generating the initial 3D model of the foot, by using cluster analysis.

In operation S1030, the foot information processing apparatus 100 may generate a final 3D model with a normal foot shape by restoring a portion of the foot that has been removed along with the ground when generating the intermediate 3D model. The foot information processing apparatus 100 may detect an outline forming the surface of the foot, from the pieces of 3D point cloud data having a preset height from the ground, among the pieces of 3D point cloud data included in the intermediate 3D model. The foot information processing apparatus 100 may generate a sole portion by vertically projecting, onto the ground, the pieces of 3D point cloud data having the preset height from the ground and corresponding to the outline. The foot information processing apparatus 100 may restore the sole portion as a portion of the foot that has been removed along with the ground. The foot information processing apparatus 100 may generate the final 3D model as a result of performing an interpolation process by applying, to the 3D model with the restored sole portion, a method of aligning normal vectors by using a virtual camera position (the position where a foot image was actually captured), or a Poisson surface reconstruction algorithm.

In operation S1040, the foot information processing unit 170 may calculate foot-related information from the final 3D model and recommend a type and size of shoes. The foot information processing apparatus 100 may calculate, from the final 3D model, the foot-related information including length information of the foot, width information of the foot, circumference information of the foot, thickness information of the foot, shape information of the foot, and instep height information of the foot. The foot information processing apparatus 100 may recommend a type and size of shoes corresponding to the foot-related information by using a deep neural network model that is pretrained to recommend a type and size of shoes by using foot-related information as an input. Here, the deep neural network model may be a model trained in a supervised learning manner by using training data including foot-related information as inputs, and types and sizes of shoes as labels.

While the present disclosure has been particularly shown and described with reference to exemplary embodiments thereof, it will be understood by those of ordinary skill in the art that various changes in form and details may be made therein without departing from the spirit and scope of the present disclosure as defined by the following claims.

According to the present disclosure, a method for measuring foot size and shape by using image processing is provided. In addition, embodiments of the present disclosure may be applied to an industrially used apparatus for measuring the inner size of an object in which an inner space is formed, by using image processing.

What is claimed is:

1. A foot information processing method executed by a processor of a foot information processing apparatus, the foot information processing method comprising:
generating an initial three-dimensional (3D) model of a foot based on a plurality of foot images, which are obtained by photographing the foot from various angles;
generating an intermediate 3D model by removing ground and noise from the initial 3D model;
generating a final 3D model with a normal foot shape by restoring a portion of the foot that has been removed along with the ground when generating the intermediate 3D model; and
calculating foot-related information from the final 3D model and recommending shoes.

2. The foot information processing method of claim 1, wherein the generating of the initial 3D model comprises:
acquiring red-green-blue (RGB) information from the plurality of foot images, which are captured while moving a camera 360 degrees around the foot;
when capturing the plurality of foot images, acquiring pieces of 3D point cloud data (a 3D point cloud set) based on information about a distance to the foot, which is measured by a light-detection-and-ranging (LiDAR) sensor; and
generating an initial 3D model of the foot by combining the RGB information with the pieces of 3D point cloud data based on the information about the distance to the foot.

3. The foot information processing method of claim 2, wherein the generating of the intermediate 3D model comprises:
detecting one or more planes by using the pieces of 3D point cloud data included in the initial 3D model, and estimating and removing one of the one or more planes as ground, based on the information about the height from the ground to the camera, which is obtained when capturing the plurality of foot images; and
removing noise generated when capturing the plurality of foot images or noise generated when generating the initial 3D model of the foot, by using cluster analysis.

4. The foot information processing method of claim 3, wherein the generating of the final 3D model comprises:
detecting an outline forming a surface of the foot, from pieces of 3D point cloud data having a preset height from the ground, among the pieces of 3D point cloud data included in the intermediate 3D model;
generating a sole portion by vertically projecting, onto ground, the pieces of 3D point cloud data having the preset height from the ground and corresponding to the outline; and
restoring the sole portion as the portion of the foot that has been removed along with the ground.

5. The foot information processing method of claim 1, wherein
the recommending of the shoes comprises: calculating, from the final 3D model, foot-related information comprising length information of the foot, width information of the foot, circumference information of the foot, thickness information of the foot, shape information of the foot, and instep height information of the foot; and recommending a type and a size of shoes corresponding to the foot-related information by using a deep neural network model that is pretrained to recommend a type and a size of shoes by using foot-related information as an input, and
the deep neural network model is a model trained in a supervised learning manner by using training data comprising foot-related information as inputs, and types and sizes of shoes as labels.

6. A computer-readable recording medium having recorded thereon a computer program for executing a foot information processing method executed by a processor of a foot information processing apparatus, the foot information processing method comprising the steps of:
generating an initial three-dimensional (3D) model of a foot based on a plurality of foot images, which are obtained by photographing the foot from various angles;
generating an intermediate 3D model by removing ground and noise from the initial 3D model;
generating a final 3D model with a normal foot shape by restoring a portion of the foot that has been removed along with the ground when generating the intermediate 3D model; and
calculating foot-related information from the final 3D model and recommending shoes.

7. The computer-readable recording medium of claim 6, wherein the step of generating the initial 3D model comprises steps of:
acquiring red-green-blue (RGB) information from the plurality of foot images, which are captured while moving a camera 360 degrees around the foot;
when capturing the plurality of foot images, acquiring pieces of 3D point cloud data (a 3D point cloud set) based on information about a distance to the foot, which is measured by a light-detection-and-ranging (LiDAR) sensor; and
generating an initial 3D model of the foot by combining the RGB information with the pieces of 3D point cloud data based on the information about the distance to the foot.

8. The computer-readable recording medium of claim 7, wherein the step of generating the intermediate 3D model comprises steps of:
detecting one or more planes by using the pieces of 3D point cloud data included in the initial 3D model, and estimating and removing one of the one or more planes as ground, based on the information about the height from the ground to the camera, which is obtained when capturing the plurality of foot images; and
removing noise generated when capturing the plurality of foot images or noise generated when generating the initial 3D model of the foot, by using cluster analysis.

9. The computer-readable recording medium of claim 8, wherein the step of generating the final 3D model comprises steps of:
detecting an outline forming a surface of the foot, from pieces of 3D point cloud data having a preset height from the ground, among the pieces of 3D point cloud data included in the intermediate 3D model;
generating a sole portion by vertically projecting, onto ground, the pieces of 3D point cloud data having the preset height from the ground and corresponding to the outline; and
restoring the sole portion as the portion of the foot that has been removed along with the ground.

10. The computer-readable recording medium of claim 6, wherein the step of recommending shoes comprises steps of:
calculating, from the final 3D model, foot-related information comprising length information of the foot, width information of the foot, circumference information of the foot, thickness information of the foot, shape information of the foot, and instep height information of the foot;
and recommending a type and a size of shoes corresponding to the foot-related information by using a deep neural network model that is pretrained to recommend a type and a size of shoes by using foot-related information as an input, and
wherein the deep neural network model is a model trained in a supervised learning manner by using training data comprising foot-related information as inputs, and types and sizes of shoes as labels.

11. A foot information processing apparatus comprising:
a processor; and
a memory operatively connected to the processor and storing at least one piece of code to be executed by the processor,
wherein the memory stores code that, when executed by the processor, causes the processor to
generate an initial three-dimensional (3D) model of a foot based on a plurality of foot images, which are obtained by photographing the foot from various angles,
generate an intermediate 3D model by removing ground and noise from the initial 3D model,
generate a final 3D model with a normal foot shape by restoring a portion of the foot that has been removed along with the ground when generating the intermediate 3D model, and
calculate foot-related information from the final 3D model and recommend shoes.

12. The foot information processing apparatus of claim 11, wherein the memory further stores code that causes the processor to,
when generating the initial 3D model, acquire red-green-blue (RGB) information from the plurality of foot images, which are captured while moving a camera 360 degrees around the foot,
when capturing the plurality of foot images, acquire pieces of 3D point cloud data (a 3D point cloud set) based on information about a distance to the foot, which is measured by a light-detection-and-ranging (LiDAR) sensor, and
generate an initial 3D model of the foot by combining the RGB information with the pieces of 3D point cloud data based on the information about the distance to the foot.

13. The foot information processing apparatus of claim 12, wherein the memory further stores code that causes the processor to, when generating the intermediate 3D model, detect one or more planes by using the pieces of 3D point cloud data, estimate and remove one of the one or more planes as ground, based on the information about the height from the ground to the camera, which is obtained when capturing the plurality of foot images, and remove noise generated when capturing the plurality of foot images or noise generated when generating the initial 3D model of the foot, by using cluster analysis.

14. The foot information processing apparatus of claim 13, wherein the memory further stores code that causes the processor to, when generating the final 3D model, detect an outline forming a surface of the foot, from pieces of 3D point cloud data having a preset height from the ground, among the pieces of 3D point cloud data included in the intermediate 3D model, generate a sole portion by vertically projecting, onto ground, the pieces of 3D point cloud data having the preset height from the ground and corresponding to the outline, and restore the sole portion as the portion of the foot that has been removed along with the ground.

15. The foot information processing apparatus of claim 11, wherein the memory further stores code that causes the processor to, when recommending the shoes, calculate, from the final 3D model, foot-related information comprising length information of the foot, width information of the foot, circumference information of the foot, thickness information of the foot, shape information of the foot, and instep height information of the foot, and recommend a type and a size of shoes corresponding to the foot-related information by using a deep neural network model that is pretrained to recommend a type and a size of shoes by using foot-related information as an input, and
the deep neural network model is a model trained in a supervised learning manner by using training data comprising foot-related information as inputs, and types and sizes of shoes as labels.
